(19) United States
(12) Patent Application Publication
(10) Pub. No.: US 2019/0055517 A1
(43) Pub. Date: Feb. 21, 2019

(54) Cell Line of M2c Macrophage and Its Applications

(71) Applicant: National Pingtung University of Science and Technology, Pingtung County (TW)

(72) Inventor: Ko-Tung CHANG, Pingtung County (TW)

(73) Assignee: National Pingtung University of Science and Technology, Pingtung County (TW)

(21) Appl. No.: 15/853,702

(22) Filed: Dec. 22, 2017

(30) Foreign Application Priority Data
Aug. 16, 2017 (TW) 106127836
Dec. 19, 2017 (TW) 106144647

(51) Int. Cl. C12N 5/0786; A61K 35/17; A61K 45/06; A61L 15/62; A61L 15/22

(52) U.S. Cl. CPC C12N 5/0645; A61K 35/17; A61K 2035/124; A61L 15/62; A61L 15/225; A61K 45/06

(57) ABSTRACT

This invention provides a cell line of M2C macrophage and its applications. The cell line is derived from monocytes isolated from bone marrows and peripheral blood. The monocytes were differentiated into M2 macrophage by macrophage colony-stimulating factor (M-CSF), and then the polarization of M2C macrophage was induced by baicalin. The MERTK, PTX3, and PD-L1 expression level of the M2C macrophage are high and promote phagocytosis. Hence it can be applied to cell therapy or biological agents of immune regulation. Also, the macrophage-conditioned medium and wound dressing prepared on the M2C cell have the effects of enhancing fibroblast proliferation and angiogenesis, which can improve wound healing in medical use, and can be applied to skin care product for skin repair and rejuvenation.

### CROSS REFERENCE TO RELATED APPLICATIONS

This application claims priority to Taiwan Invention Patent Application No. 106127836 filed on Aug. 16, 2017, and Taiwan Invention Patent Application No. 106144647 filed on 19 Dec. 2017, the disclosure of which is hereby Incorporated in its entirety by reference.

### FIELD

The invention relates to a cell line of M2C macrophage called NPUST-M2φ-1 (deposited in the CCTCC with accession number Mxxxxx) and its applications. More specifically, the invention relates to the M2C macrophage differentiated from M2 macrophage by baicalin induction, wherein the M2 macrophage is derived from monocytes by macrophage colony-stimulating factor (M-CSF). Also, the invention relates to the macrophage-conditioned medium and wound dressing prepared on the cell line.

### BACKGROUND

Occurrence of acute coronary syndrome and other thrombosis diseases is related to mechanisms of blood clots formation. The blood clots form after the vascular endothelial cell are damaged, when vascular smooth muscle cell and extracellular matrix form a fibrous cap to wrap around the necrotic core of dead foam cells after metabolizing lipid. Along with the apoptosis of endothelial cells and increase of matrix metalloproteinases, fibrous cap becomes thin and causes rupture of dots. This leads to more severe damage of vascular intima and eventually causes thrombosis.

Rupture of blood clots accelerates cell apoptosis. When efferocytosis, a process by which apoptotic cells are removed by phagocytic cells, cannot be sufficiently executed, the remaining apoptotic cells undergo postapoptotic cellular necrosis. It will initiate apoptotic cells to release substances promoting inflammation and thrombosis, hence causing chronic tissue inflammation and autoimmune diseases.

Mertk (MER proto-oncogene, tyrosine kinase) gene affects efferocytosis by regulating the conjugation of macrophages and the phosphatidylserine of apoptotic cells. Studies have shown that in the mutated murine model which did not express Mertk gene, the apoptotic cells accumulate and necrotize. Studies have also shown that the deficiency in this efferocytosis receptor will accelerate atherosclerosis. (Thorp, Edward, et al. 2008) In other aspect, the protein expressed by PTX3 (Pentraxin 3) can combine with apoptotic cells, causing immature dendritic cells failed to use cell remnant for antigens-presentation. This helps macrophages to phagocytose apoptotic cells and prevent autoimmunity reaction caused by antigen-presenting cells. (Rovere, Patrizia, et al. 2000.) In prior art it has been disclosed that using biotinylated long fragmented PTX3 to treat apoptotic cells can prevent apoptotic cells from combining with immature dendritic cells.

Furthermore, PD-1 (program death-1) protein expresses in activated T cell and B cell; its ligand PD-L1 mainly expresses in macrophages. When PD-L1 conjugates with PD-1 from activated T cells, functions of cytotoxic T cells are inhibited. By deactivating T cell, PD-L1 promotes peripheral tissue autoimmune tolerance and maintaining immune tolerance balance. Based on previous studies, a technical mean to promote phagocytosis, such as efferocytosis, is expected in the field of human medicine and animal medicine, in order to further improve chronic inflammation and autoimmune related diseases.

Many extracts from Chinese herbs have been proved to inhibit immunity inflammation. Previous studies have shown that in rabbit model tests, extracts from Salvia miltiorrhiza and Andrographis paniculata can mitigate blood clots accumulating and symptoms of atherosclerosis. However, the mechanisms of how these Chinese herbal ingredient intervene with blood clot formation and efferocytosis remain unclear.

Normal wound healing process includes different clinical phases such as hemostasis, inflammation, proliferation, and maturation. Wounds require appropriate treatment throughout the different phases of healing in order to regulate the interaction between different cells, cytokines and extracellular matrix. For example, skin often takes the brunt of injury, and the healing of epidermal wound consists of re-epithelialization, granulation tissue proliferation and collagen synthesis.

Macrophages and fibroblasts play important roles respectively in multiple healing phases. During the proliferation phase, fibroblasts proliferate and granulation tissues are formed on the wound, wherein part of these cells are further differentiated into myofibroblast and produce extracellular matrix mainly composed of collagen. Meanwhile, when a wound is formed and repaired, macrophages are activated into M1 and M2 macrophages respectively. M1 macrophages promote inflammation during inflammation phase, whereas M2 macrophages produce non-inflammatory cytokines, assist tissue repair and angiogenesis (Ploeger, Diana T A, et al., 2013).

Although human body can self-repair wounds, wound dressings can further protect the wound during healing process, which provide protection against irritants and reduce wound infection. Traditionally, dressings like gauzes and artificial skins are used; due to development of biomaterial technology in recent years, biomaterials including alginates, hydrocolloids, collagen, polyurethane, pectin, hyaluronic acid and silk protein are also used for wound dressings (Gil, Eun Seok, et al., 2013).

However, the aforementioned biotic material dressings mainly only give the wound a suitable healing environment; more specifically, such as keeping the wound moist and bacteria-free, absorbing exudate and preventing adhesion. The wound dressing itself does not directly participate in the wound healing mechanism, which would promote cell proliferation or tissue repair in wounds.

### REFERENCES CITED

### Foreign Patent Documents

WO 2002/036151 World Intellectual Property Organization

### Other Publication

Anand, Rahul J., et al. “Toll-like receptor 4 plays a role in macrophage phagocytosis during peritoneal sepsis.” Journal of pediatric surgery 42.6 (2007): 927-933.
Gil, E. S., Panilaitis, B., Bellas, E., and Kaplan, D. L. (2013). Functionalized silk biomaterials for wound healing. Advanced healthcare materials, 2(1), 206-217.
Ploeger, D. T., Hosper, N. A., Schipper, M., Koerts, J. A., de Rond, S., and Bank, R. A. (2013). Cell plasticity in wound healing: paracrine factors of M1/M2 polarized macrophages influence the phenotypical state of dermal fibroblasts. Cell Communication and Signaling, 11(1), 29.
Rovere, P., Peri, G., Fazzini, F., Bottazzi, B., Doni, A., Bondanza, A., Zimmermann V. S., Garland C., Fascio U., Sabbadini M. Z., Rugarli C, Mantovani A., and Manfredi, A. A. The long pentraxin PTX3 binds to apoptotic cells and regulates their clearance by antigen-presenting dendritic cells. Blood 96.13 (2000): 4300-4306.
Thorp, E., Cui, D., Schrijvers, D. M., Kuriakose, G., and Tabas, I. 2008. Mertk receptor mutation reduces efferocytosis efficiency and promotes apoptotic cell accumulation and plaque necrosis in atherosclerotic lesions of apoe−/− mice. Arteriosclerosis, thrombosis, and vascular biology. 28.8: 1421-1428.
Li, Y. J., Zhu, X. X., Yang, Q., Weng, X. G., and Chen, Y. 2011. Effect of Salviae Miltiorhize Radix and Andrographitis Herba extracts on vessel pathological changes and lipid metabolism in atherosclerosis rabbits. Chin Tradit Herb Drugs. 42: 760-4.

### SUMMARY

This invention aims to provide a technical mean that can simultaneously increase MERTK, PTX3 and PD-L1 expression level of macrophage, promote efferocytosis and other phagocytosis activities, and improve chronic inflammation and other autoimmune diseases.

In another aspect, this invention also aims to provide a wound dressing comprising macrophage-conditioned medium that can directly participate in the wound healing mechanism and promote cell proliferation or tissue repair.

The inventors of the present invention carry out in-depth studies in view of the foresaid issues. Monocytes were first separated from bone marrows and peripheral blood, then use M-CSF (Macrophage colony-stimulating factor) to differentiate monocytes into M2 macrophages. Lastly, use Baicalin to induce the M2 macrophages to differentiate and obtain the macrophage cell line NPUST-M2φ-1 (deposited in CCTCC with accession number Mxxxxx) with high expression levels of MERTK, PTX3, PD-L1 and IL-10. Those macrophage cell lines can promote phagocytosis and be used in cell therapy or drugs to improve chronic inflammation and autoimmune related diseases. Meanwhile, the conditioned medium and dressing created from this macrophage can promote fibroblast proliferation, angiogenesis, wound healing, cells proliferation or tissue repair.

Therefore, this invention provides techniques comprising:
1. A M2C macrophage cell line called NPUST-M2φ-1, deposited in CCTCC (China Center for Type Culture Collection) with accession number Mxxxxx, wherein the M2C macrophage cell is derived from monocytes collected from bone marrows or peripheral blood, and differentiated Into M2 macrophage using M-CSF (macrophage colony-stimulating factor), then further induced to differentiate into M2C macrophage using baicalin.
2. A method to prepare a M2C macrophage cell line, comprising:
   Step (a): Collecting monocytes from bone marrow and peripheral blood;
   Step (b): Using M-CSF to induce M2 macrophage polarization of monocytes;
   Step (c): Using baicalin to induce M2C macrophage polarization of M2 macrophage.
3. A method of promoting phagocytosis by cell therapy, wherein the M2C macrophage of claim 1 is used in the said cell therapy.
4. A method of regulating T cell immune by cell therapy, wherein the M2C macrophage of claim 1 is used in the said cell therapy.
5. A method of regulating VEGF gene by cell therapy, wherein the M2C macrophage of claim 1 is used in the said cell therapy.
6. A method of promoting phagocytosis using cell preparation, wherein the cell preparation comprises M2C macrophage of claim 1.
7. A method of regulating T cell immune regulation using cell preparation, wherein the cell preparation comprises M2C macrophage of claim 1.
8. A method of regulating VEGF gene using cell preparation comprises M2C macrophage of claim 1, wherein the VEGF gene is selected from the group of VEGF-A, VEGFR-1 and VEGFR-2.
9. A method of improving mammal immune diseases by cell therapy, wherein the M2C macrophage of claim 1 is used in the said cell therapy,
10. A method of improving mammal immune diseases using cell preparation, wherein the cell preparation comprises M2C macrophage of claim 1.
11. A baicalin-M2C macrophage-conditioned medium that improves wound healing, which is obtained after culturing baicalin-induced M2C macrophage in basal medium for 4 hours.
12. The baicalin-M2C macrophage-conditioned medium of claim 11, wherein the said basal medium is X-VIVO-10.
13. A method of preparing M2C macrophage conditioned medium which improves wound healing, comprising the steps of:
   Step (a): Using baicalin to induce M2C polarization of M2 macrophage;
   Step (b): Using the basal medium to incubate M2C macrophage;
   Step (c): Collecting baicalin-M2C macrophage conditioned medium.
14. The method of claim 13, wherein the said step (a) includes incubating M2 macrophage in medium containing 50 μM baicalin for 24 hours.
15. The method of claim 13, wherein the said step (b) includes using X-VIVO-10 medium as basal medium and incubating M2C macrophage for 4 hours.
16. A wound dressing that improve wound healing, wherein the dressing comprises the baicalin-M2C macrophage conditioned medium of claim 11.
17. A method of preparing wound dressings, comprises the steps of:
   Step (a): Using baicalin to induce M2C polarization of M2 macrophage;
   Step (b): Incubating the aforementioned M2C macrophage in basal medium;
   Step (c): Collecting baicalin-M2C macrophage-conditioned medium;
   Step (d): Using a dressing substrate to absorb baicalin-M2C macrophage-conditioned medium.
18. The method of claim 17, wherein the said dressing substrate of step (d) is polyvinyl alcohol (PVA) hydrogel.
19. A method of promoting fibroblast proliferation and angiogenesis using macrophage conditioned medium, wherein the said macrophage conditioned medium is baicalin-M2C macrophage-conditioned medium obtained after culturing baicalin-induced M2C macrophage in basal medium for 4 hours.
20. A method of promoting wound healing and tissue repair using macrophage conditioned medium, wherein the said macrophage conditioned medium is baicalin-M2C macrophage-conditioned medium obtained after culturing baicalin-induced M2C macrophage in basal medium for 4 hours.

There is no specific restriction on methods of collecting monocytes from peripheral blood. For example, both AMD3100 and G-CSF reagent can be used based on requirements.

The immune regulation of T cell mentioned in this invention has no specific restrictions. For example, using M2C macrophage cell line with high PD-L1 expression level from this invention to inhibit cytotoxic T cells function and promote self-immune tolerance in peripheral tissue.

The regulation of VEGF gene mentioned in this invention has no specific restrictions. For example, using M2C macrophage cell line from this invention to enhance the expression level of gene VEGF-A and its receptor VEGFR-1, VEGFR-2.

The immune diseases mentioned in this invention have no specific restrictions. For example, it can be atherosclerosis, arteriosclerosis, multiple sclerosis, amyotrophic lateral sclerosis (ALS), systemic lupus erythematosus, systemic sclerosis, atopic dermatitis, autoimmune hepatitis, rheumatoid arthritis, hemolytic anemia, inflammatory bowel disease, chronic inflammatory disease and gingivitis-stomatitis-pharyngitis complex.

The baicalin induced differentiated M2C macrophage cell line NPUST-M2φ-1 in this invention has higher MERTK and PTX3 expression level compared to LPS, IL-4 induced macrophages in prior art. It can promote efferocytosis or phagocytosis, lessen accumulation of apoptotic cells and improve chronic inflammatory or autoimmune diseases.

Meanwhile, the PD-L1 expression level of M2C macrophage call line NPUST-M2φ-1 is also high. This can inactivate T cell, promote tissue autoimmune tolerance, maintain the balance of immune tolerance and improve autoimmune related diseases.

Moreover, the expression levels of VEGF-A and its receptor VEGFR-1, VEGFR-2 in M2C macrophage provided by this invention are also high, which can promote functions of VEGF gene family, such as angiogenesis by autocrine regulation mechanism.

Furthermore, the baicalin-M2C macrophage-conditioned medium provided by this invention contains specific cytokines such as IL-10 and VEGF-A which can enhance proliferation of dermal fibroblast and angiogenesis, hence accelerate the wound healing and tissue repair process. This baicalin-M2C macrophage-conditioned medium can further be used in medical field such as wound dressing and membrane in dental surgery application to improve wound healing. Moreover, this invention can also be used in skin care products that needs repair or regeneration feature.

### BRIEF DESCRIPTION OF THE DRAWINGS

FIG. 1 shows a flow cytometry analysis illustrating the survival rate of thawed M2C macrophage after cryopreservation.

FIG. 2 shows scattered plot and bar chart of flow cytometry analysis result of macrophage molecular marker CD11b, M2 molecular marker CD206 and M1 molecular marker CD80 (treated by M-CSF).

FIG. 3 shows scattered plot and bar chart of flow cytometry analysis result of M2 molecular marker CD206, M1 molecular marker CD86. (Treated by LPS, IL-4 and baicalin).

FIG. 4 is an image of macrophage cell morphology under microscope.

FIG. 5 illustrates the comparative gene expression level of TNF-Alpha, IRF5, and IRF4 in embodiment 1, comparison 1 and comparison 2.

FIG. 6 illustrates the comparative gene expression level of Arginase-1, 1110, and IL-6 in embodiment 1, comparison 1 and comparison 2.

FIG. 7 illustrates the comparative gene expression level of PTX-3, Mertk in embodiment 1, comparison 1 and comparison 2.

FIG. 8 illustrates the comparative gene expression level of VEGF-A in embodiment 1, comparison 1 and comparison 2.

FIG. 9 shows comparative gene expression level of VEGF-A, VEGF-B and VEGF-D between M1 and M2 macrophage (Raw264.7 derived) after treating baicalin.

FIG. 10 shows comparative gene expression level of VEGFR1, VEGFR2 and VEGFR 3 between M1 and M2 macrophage (Raw264.7 derived) after treating baicalin.

FIG. 11 shows comparative gene expression level of VEGFR1, VEGFR2 and VEGFR 3 between baicalin treated macrophage (Raw264.7 derived) and IL-4 treated macrophage (Raw264.7 derived).

FIG. 12 shows comparative gene expression level of VEGFR1, VEGFR2 and VEGFR 3 between M1 and M2 macrophage (Raw264.7 derived) after treating baicalin.

FIG. 13 illustrates comparative VEGF-A protein expression level of macrophage based on ELISA result.

FIG. 14 illustrates comparative MERTK protein expression level of macrophage based on flow cytometry result.

FIG. 15 illustrates average MERTK protein expression level of single macrophage cell based on mean fluorescent intensity of flow cytometry result.

FIG. 16 shows histogram and bar chart showing the percentage of macrophages undergoing phagocytosis based on flow cytometry result.

FIG. 17 shows histogram and bar chart showing percentage of macrophages undergoing phagocytosis which also express MERTK, based on flow cytometry result.

FIG. 18 shows microscope and fluorescence microscope images of macrophages phagocytosing FITC-beads.

FIG. 19 shows percentage of apoptotic cells derived from Raw264.7 cell line under different concentration of H₂O₂.

FIG. 20 is a scatter plot based on flow cytometry analysis to examine apoptotic cells and efferocytosed cells. P4 region are apoptotic cells obtained after induced by H₂O₂ (1.5 mM) and dyed with FITC-CFSE fluorescent dye; P5 region are differentiated M2C macrophage induced by baicalin and dyed with PE-MERTK; P6 region are macrophage which undergo apoptotic cells efferocytosis and simultaneously express FITC and PE fluorescent light, which means they are macrophages both MERTK-positive and CFSE-positive.

FIG. 21 shows average CFSE protein expression level and average MERTK protein expression level of single macrophage cell undergo efferocytosis, based on mean fluorescent intensity of flow cytometry result.

FIG. 22 is a flow cytometry analysis result showing the phenotype of T cell in murine peripheral blood, spleen and bone marrow after injecting M2C macrophage.

FIG. 23 shows microscopic image of the growth of dermal fibroblasts in IL4-M2 macrophage conditioned medium.

FIG. 24 shows microscopic image of the growth of dermal fibroblasts in baicalin-M2C macrophage conditioned medium.

FIG. 25 shows dermal fibroblasts proliferation in IL4-M2 macrophage conditioned medium based on flow cytometer analysis result.

FIG. 26 shows dermal fibroblasts proliferation in balcalin-M2C macrophage conditioned medium based on flow cytometry analysis result.

FIG. 27 shows illustration of wound healing of mice after applying dressing which absorbed baicalin-M2C macrophage conditioned medium.

### DETAILED DESCRIPTION

The present invention will be further exemplified by the following examples, which are not to be seen as limiting. The embodiments and description are used for Illustrating the details and effect of the present invention.

### [Induced Polarization of M2C Macrophage]

Use PBS containing 0.5% BSA to flush out bone marrow cells from murine (C57BL/6J, 8-20 weeks, Taiwan National Laboratory Animal Center) femur, then use Ficoll-Hypaque (GE healthcare, SWEDEN) to isolate monocytes.

Then, incubate 2×10⁶ monocytes in 50 ng/ml M-CSF contained RPMI 1640 (10% FCS) medium under 37° c., 5% CO₂ for seven days to induce M2 macrophage polarization. On the eighth day, collect 2×10⁵ M2 macrophage and incubate them in RPMI 1640 (10% FCS) medium with Baicalin (50 μM, Tokyo Chemical Industry, JAPAN) for 24 hours. Baicalin will induce M2 macrophage to differentiate into M2C macrophage.

### [Cryopreservation of M2C Macrophage]

Remove the cell culture medium and wash with PBS for two times. Add 1 ml 0.025% trypsin (Biowest, Missouri, USA) and incubate cell in incubator for cells to stop adhesions. After five minutes incubation, use microscope to confirm that more than 90% of cells have been detached, then immediately transfer the cells to 15 ml centrifuge tube and add into the same volume of RPMI 1640 (10% FCS) medium as trypsin to terminate trypsin reaction. Next, add 4 ml PBS into the petri dish to collect remnant cells and transfer them into the same centrifuge tube. Centrifuge cells at 4° C. for 5 mins at rotating speed of 500×g. Meanwhile, add 10% dimethyl sulfoxide (DMSO) (Sigma-Aldrich, USA) into RPMI 1640 (10% FCS) medium to prepare cryopreservation medium. After centrifugation, remove the supernatant and place the cells in 1 ml of the aforementioned cryopreservation medium for suspension and dilution. Next, transfer the cells to cryo tubes (Thermo Fisher, Denmark) and immediately place the cryo tubes into −80° C. freezers filled with 95% ethanol to freeze overnight. After freezing, preserve the frozen cryo tubes in liquid nitrogen tank.

During thawing process, take out the cells from liquid nitrogen and place it in 37° c. sterilized water bath to defreeze. After thawing cells, transfer cells to a 15 ml centrifuge tube containing 9 ml cold PBS and centrifuge cells at 4° C. for 5 mins at rotating speed of 500×g. Next, remove the supernatant and add 10 ml PBS to wash the cells. Centrifuge cells again at 4° C. for 5 mins at rotating speed of 500×g and remove the supernatant. Suspend the cells using fresh medium (90% RPMI+10% FCS). Finally, seed the cells in petri dish and incubate cell with condition of 37° C., 5% CO₂.

The survival percentage of cells after thawing is shown in FIG. 19. Results were analysed by flow cytometry, observed with forward scatter (FSC) and dyed with Propidium Iodide (PI). According to division of the results, P4 population are survival cells, which has 99% of survival rate before cryopreservation and 84% after cryopreservation.

### [Preparation of the Cell-Conditioned Medium]

Use PBS to wash baicalin Induced differentiated M2C macrophages and culture the cells in X-VIVO-10 medium (Lonza Group, Switzerland) for 4 hours. After 4 hours, collect baicalin-M2C macrophage conditioned medium and preserve it at −20° C.

Moreover, the preparation of IL4-M2 macrophage conditioned medium mentioned in examples below is prepared by replacing baicalin induced differentiated M2C macrophage by IL-4 induced differentiated M2 macrophage. The remaining steps are same as above preparation protocol.

### [Preparation of Hydrogel Dressing]

To produce hydrogels used for absorbing conditioned medium, mix polyvinyl alcohol (PVA, average molecular weight 146,800-186,000, +99% hydrolyzed, Sigma-Aldrich, USA) with dextran (average molecular weight 60,000-90,000) and undergo freeze-thaw (FT) cycle. More specifically, based on the proportion in the chart 1 below, dissolve PVA and dextran in distilled water respectively. Next, mix gentamicin with the PVA/dextran solution in vortex for 1 hour and then pour into petri dishes. Place the petri dishes in −20° C. freezer for 18 hours, and then thaw them at 25° C. for 6 hours. Repeat the freeze-thaw cycle 3 times to produce the polyvinyl alcohol (PVA) hydrogel.

CHART 1

| Ingredient | | |
|---|---|---|
| (Proportion in 100 g of hydrogel) | g | % |
| PVA (g) | 2.5 | 2.5 |
| Dextran (g) | 1.13 | 1.13 |
| Gentamicin (g) | 0.1 | 0.1 |
| Water (mL) | 100 | 100 |

### [Preparation of Hydrocolloids Contains Baicalin-M2C Macrophage-Conditioned Medium]

Dip hydrogels into balcalin-M2C macrophage-conditioned medium and IL4-M2 macrophage conditioned medium respectively. Shake under 100 rpm for 24 hours then carefully take out and wash with PBS for 5 times.

### EXAMPLES

The examples below are used for Illustrating the objectives and effects of the present invention, which are not to be seen as limiting.

### Example 1: [Murine M2C Macrophage Differentiation Induction]

Embodiment 1 is prepared by the steps described above, isolate monocytes from murine bone marrow and use M-CSF to induce differentiation of M2 macrophage, and then use baicalin to induce M2C macrophage polarization. In comparison 1, incubate murine M2 macrophage in RPMI 1640 (10% FCS) containing LPS (lipopolysaccharide, 1 μg/ml, Sigma-Aldrich) for 2 hours. In comparison 2, incubate murine M2 macrophage in medium containing IL-4 (10 Cell Guidance System, Cambridge, UK) for 24 hours. The control group of M2 macrophage is incubated in RPMI 1640 (10% FCS) without any drugs. The conditions are shown below in chart 2.

CHART 2

| | Embodiment 1 | Comparison 1 | Comparison 2 |
|---|---|---|---|
| Drugs to induce | Baicalin | LPS | IL-4 |
| differentiation | | | |
| Concentration | 50 μM | 1 μg/ml | 10 ng/ml |
| Incubation time | 24 hours | 2 hours | 24 hours |

After induced differentiation is complete, use flow cytometry to analyse the macrophage phenotype, in the meantime, also use real time PCR (qPCR) to observe mRNA expression level of each gene.

TNF-Alpha: M1 macrophage expression gene
IRF5: M1 macrophage polarization promoting gene
IRF4: M2 macrophage polarization promoting gene
ARG1 (Arginase-1): M2a macrophage molecular marker
IL-10: cytokine that promotes M2 macrophage polarization and inhibits inflammation
IL-6: cytokine that promotes inflammation and also serves as M2b macrophage molecular marker.
MERTK: a gene that serves as M2C macrophage molecular marker and promotes phagocytosis
PTX3: a gene that serves as M2C macrophage molecular marker and promotes phagocytosis
VEGF-A: vascular endothelial growth factor
PD-L1: a gene that regulates T cell and promotes tissue autoimmune tolerance

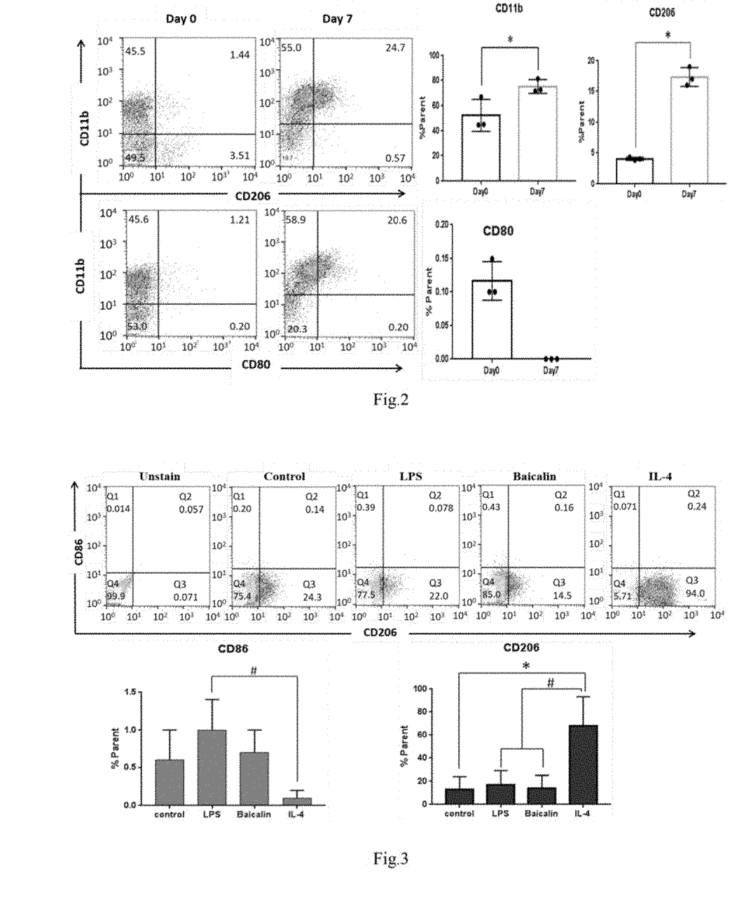

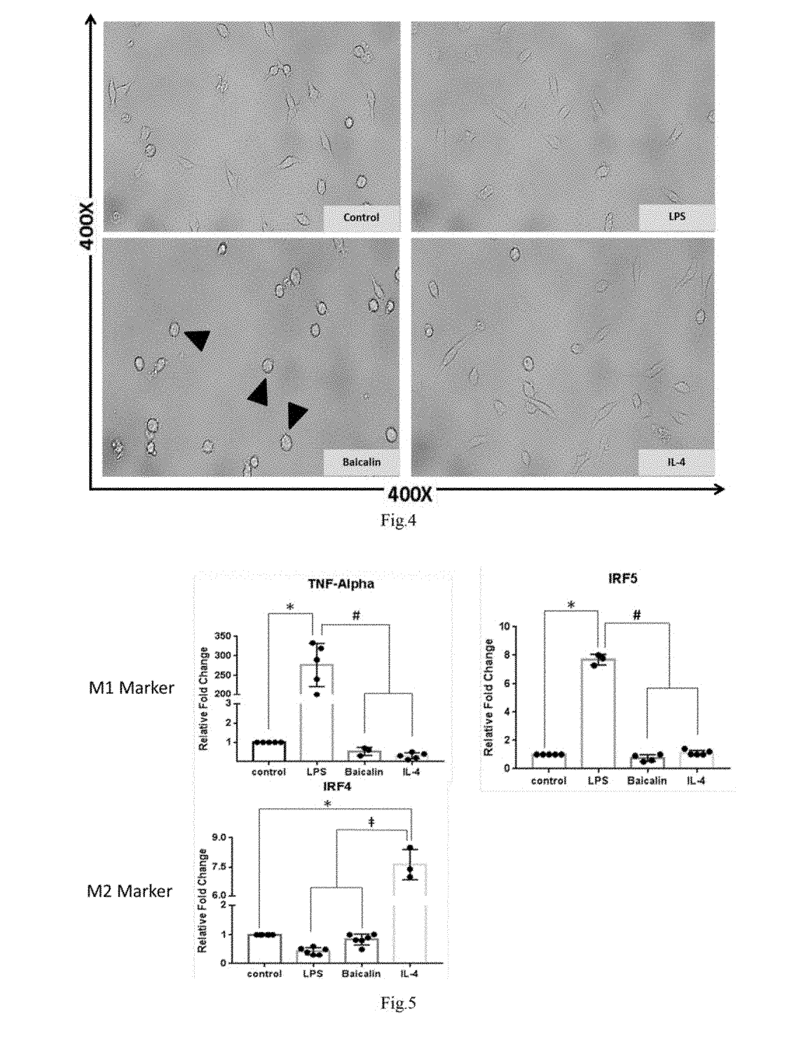

FIG. 2˜4 show results of macrophage phenotypes analysed by flow cytometry. After monocytes were treated with M-CSF, as shown in FIG. 2, the cells that were treated for 7 days have the highest percentage of expressing molecular marker CD11b (common marker for macrophage) and CD206 (common marker for M2 macrophage) at the same time (CD11b+CD206+, *p<0.05). Treated with LPS, IL-4 and baicalin respectively, as shown in FIG. 3, comparison 1 has higher percentage of M1 macrophage (C086+CD206−, #p<0.05) and comparison 2 has higher percentage of M2 macrophage (CD86−CD206+,*p<0.05). On the contrary, the percentage of cells expressing CD86 and CD206 in embodiment 1 was lower than both comparison 1 and 2 (#p<0.05). Meanwhile, in microscopy image (FIG. 4), unlike the control group treated with M-CSF and M2 macrophages in comparison 2, morphology of macrophages in embodiment 1 (marked by arrow) appeared to be ellipse instead of spindle shaped. This result shows that the phenotype and morphology characteristics of baicalin induced macrophages were different from typical M2 macrophages.

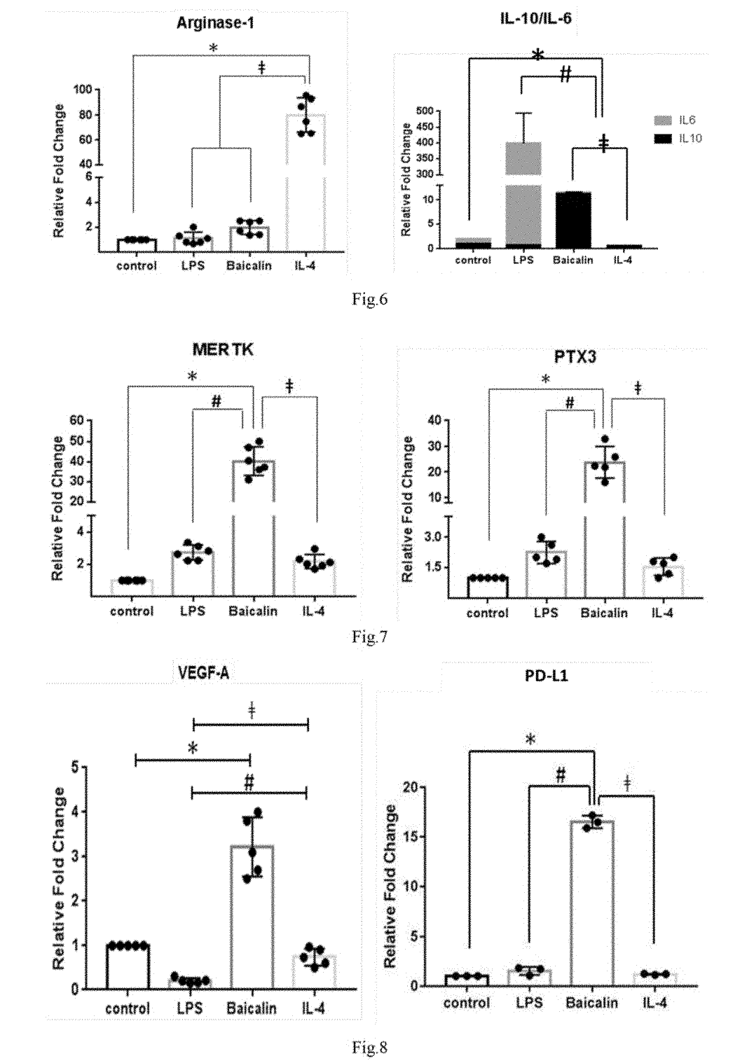

The mRNA expression level in each gene is shown in FIG. 5˜FIG. 8. FIG. 5 shows that in embodiment 1, the expression level of TNF-Alpha, IRF5 gene that promotes M1 macrophage polarization are significantly lower than comparison 1 (#p<0.05), while the expression level of gene IRF4 that promotes M2 macrophage polarization is also significantly lower than comparison 2 (*p<0.05). Furthermore, as shown in FIG. 6, ARG1 expression level in embodiment 1 was significantly lower than comparison 2 (*p<0.05), IL-6 expression level was also low, but the IL-10 expression level was significantly higher than comparison 1 and 2 (*: compared to control group. #: compared to LPS treated group. ‡: compared to IL-4 treated group. P<0.05). Meanwhile, as shown in FIG. 7 and FIG. 8, expression level of VEGF-A, PD-L1, MERTK, PTX3 were all significantly higher than control, comparison 1 and 2 (*: compared to control group. #: compared to LPS treated group. ‡: compared to IL-4 treated group. P<0.05).

This result shows that comparing to the drugs treatment in each comparison, M2 macrophages treated with baicalin have high expression level of phagocytosis related genes MERTK and PTX3, which can promote phagocytosis in immune system. Also, the showed high expression level of PD-L1 can help regulate T-cell immune system, promote tissue autoimmune tolerance and further be applied in cell therapy.

In the meantime, anti-inflammatory gene IL-10 and vascular endothelia growth factor (VEGH-A) expression levels of M2 macrophages treated with baicalin are significantly higher, whereas IL-6 expression level is significantly lower than comparison 1 (p<0.05). IL-6 is a proinflammatory cytokine that can promote inflammation and induce M1 polarization, whereas IL-10 is an anti-inflammatory cytokine that induce M2 polarization. Moreover, VEGF-A is vascular endothelial growth factor that is highly associated with wound healing, which can promote angiogenesis and hence the regenerated blood vessels can provide necessary oxygen and nutrition for wound healing process. Therefore, M2 macrophages treated with baicalin can promote angiogenesis and inhibit inflammation during wound healing process.

### Example 2: Induction of VEGF Family and PD-L1 Gene Expression in Murine Macrophage

In this example, experiment was performed to observe if baicalin can induce expression of VEGF family and PD-L1 in M1 and M2 macrophage derived from Raw264.7 cell line (ATCC, USA). M1 macrophage obtained from treating Raw264.7 macrophage with LPS (1 μg/ml, Sigma-Aldrich) for 2 hours, and M0 macrophage (control group) cultured in simple RPMI 1640 (10% FCS) medium, are respectively cultured in RPMI 1640 (10% FCS) medium with either Baicalin (50 μM, Tokyo Chemical Industry, Japan) or IL-40 (10 ng/ml, Cell Guidance System, Cambridge, UK). Collect the cells after 24/48/72 hours of cell culturing. Then observe the mRNA expression level of VEGF family, VEGF receptor and PD-L1 using reverse transcription-real time PCR (RT-qPCR).

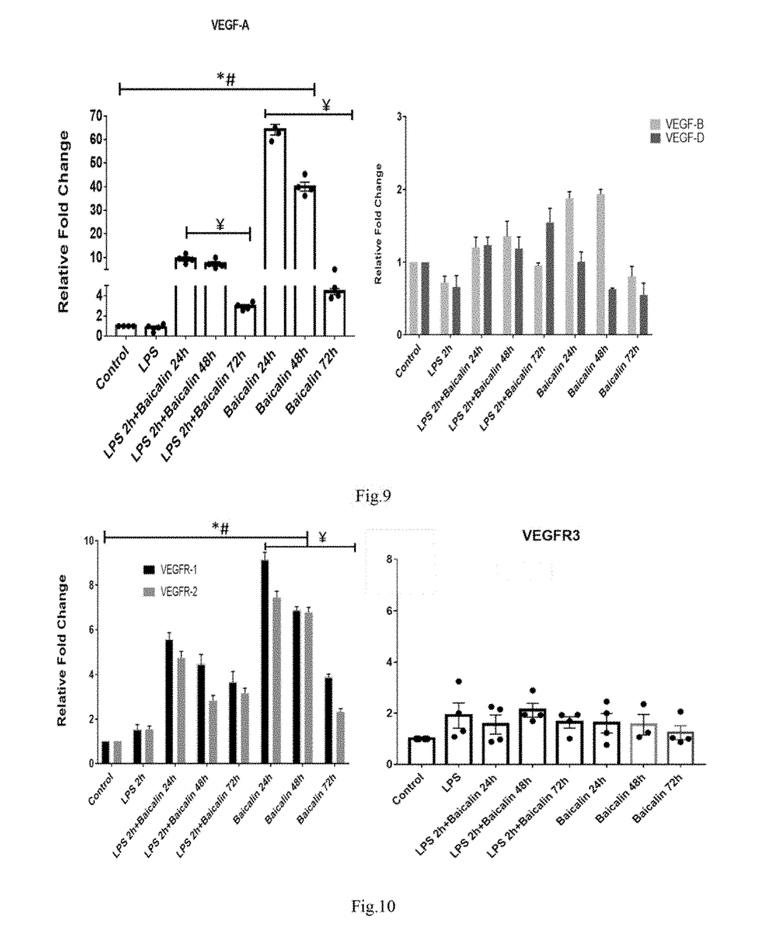

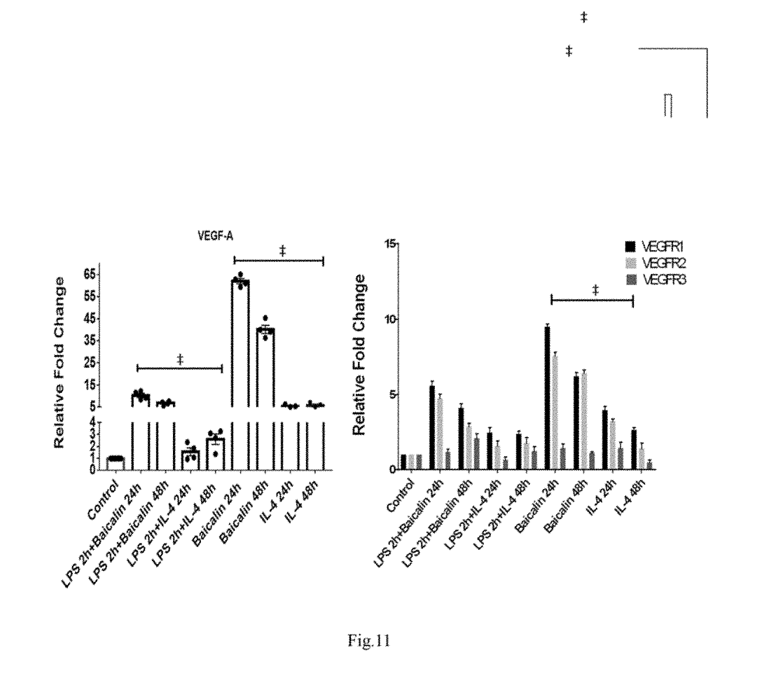

The mRNA expression level of VEGF family and VEGF receptor are shown in FIG. 9˜FIG. 11. FIG. 9 illustrates that all the groups treated with baicalin has higher level of VEGF-A than M1, M2 macrophages without balcalin treatment (LPS, control). Expression levels of different VEGF receptors are shown in FIG. 10, VEGFR-1 and VEGFR-2 expression levels are higher in those treated with baicalin (*: compared to control group, #: compared to LPS treated group, ‡: compared to IL-4 treated group). Furthermore, when comparing baicalin treated group and IL-4 treated group with the same treatment duration, as shown in FIG. 11, VEGF-A, VEGFR1, VEGFR2 mRNA expression levels were all significantly higher in baicalin treated group than IL-4 treated group (‡: compared to IL-4 treated group, p<0.05).

Results show that even for the differentiated M1 macrophages, baicalin can still promote M2C polarization and enhance VEGF-A, VEGF receptor expression level effectively greater than IL-4. Meanwhile, based on the enhancement of VEGF receptor expression, the baicalin induced differentiated M2C macrophages can perform VEGF gene autocrine regulation.

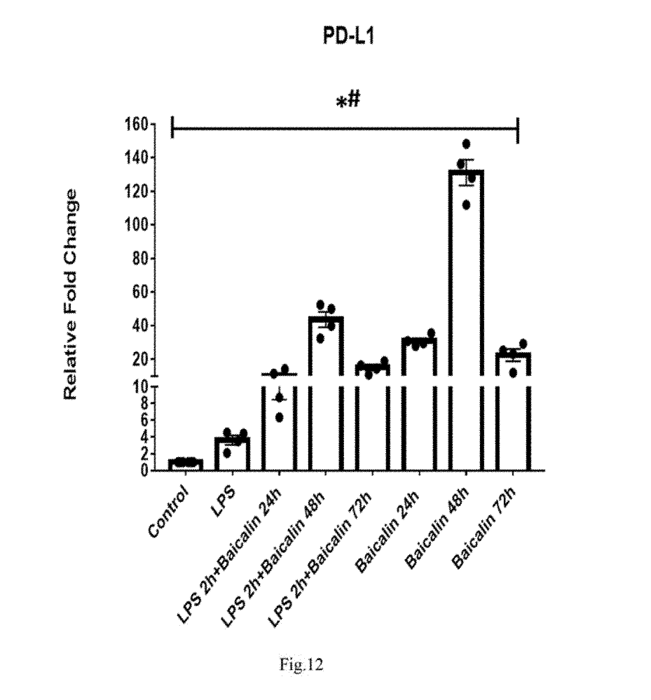

Moreover, expression level of PD-L1 shown in FIG. 12 indicates that all the groups treated with balcalin have higher PD-L1 expression levels than M1, M2 macrophages without balcalin treatment (*: compared to control group, #: compared to LPS treated group, p<0.05). This result shows that M2 macrophages (derived from Raw264.7 cell line) treated with baicalin can participate in T cell immune regulation, balance immune tolerance regulation and further be used in cell therapy.

### Example 3: VEGF-A Protein Secretion Promoted by Baicalin in Macrophage

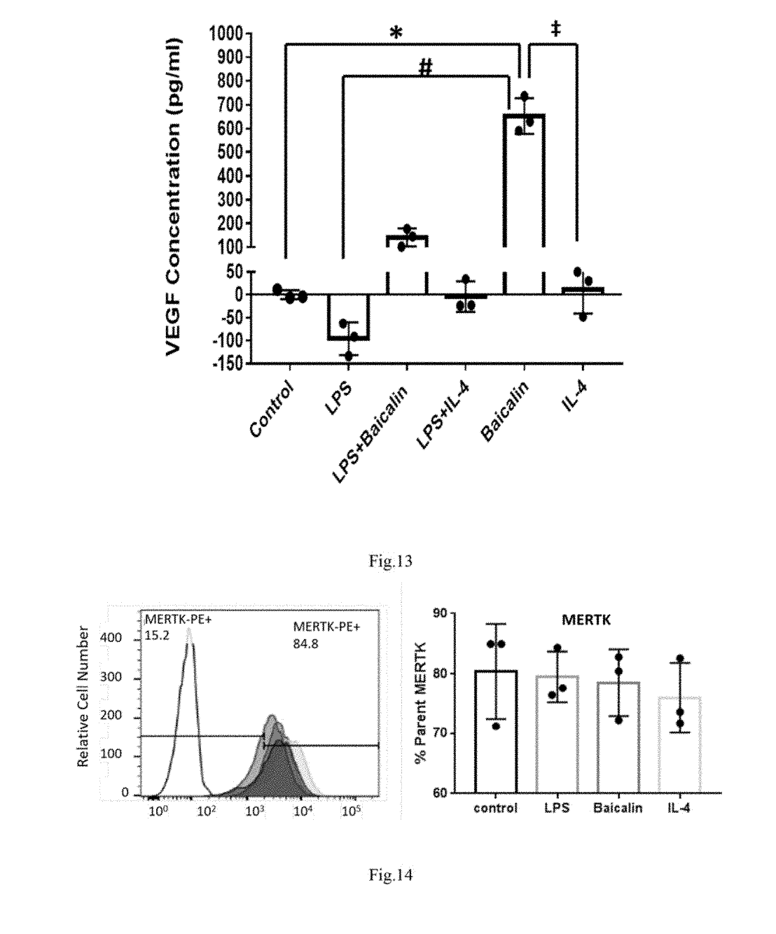

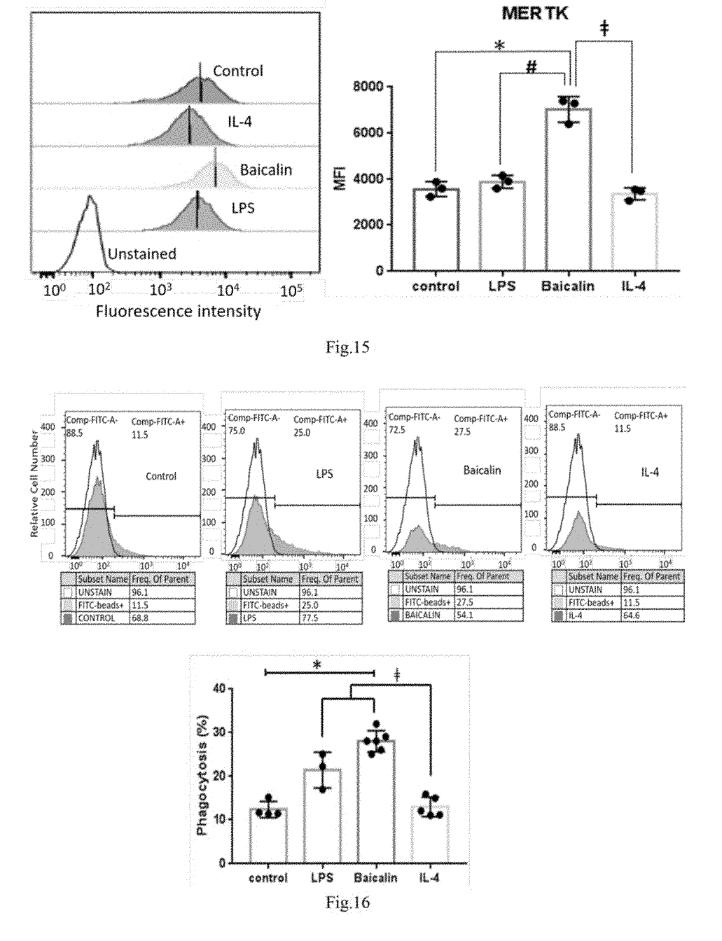

In this example, experiment was performed to further examine whether M2C cells treated with baicalin will express VEGF-A protein. Add LPS (1 μg/ml), baicalin (50 μM) and IL-4 (10 ng/ml) respectively to medium culturing 5×10; Raw264.7 macrophages (ATCC, USA) and culture for 48 hours. Collect the conditioned medium and quantify VEGF-A protein by EUSA (Mouse VEGF EUSA Kit, Code: EK0541, BosterBio, CA, USA). Control group is cultured in the medium (90% DMEM+10% FBS) without any mentioned drugs. Result is shown in FIG. 13, the VEGF-A protein detected in baicalin-M2C macrophage conditioned medium is higher than control group and other groups treated with other drugs (*: compared to control group, #: compared to LPS treated group, ‡: compared to IL-4 treated group, p<0.05). M2C macrophage treated with baicalin can promote macrophage to secrete VEGF-A protein and the efficacy is greater than IL-4.

### Example 4: MERTK Protein Secretion Promoted by Baicalin in Macrophages

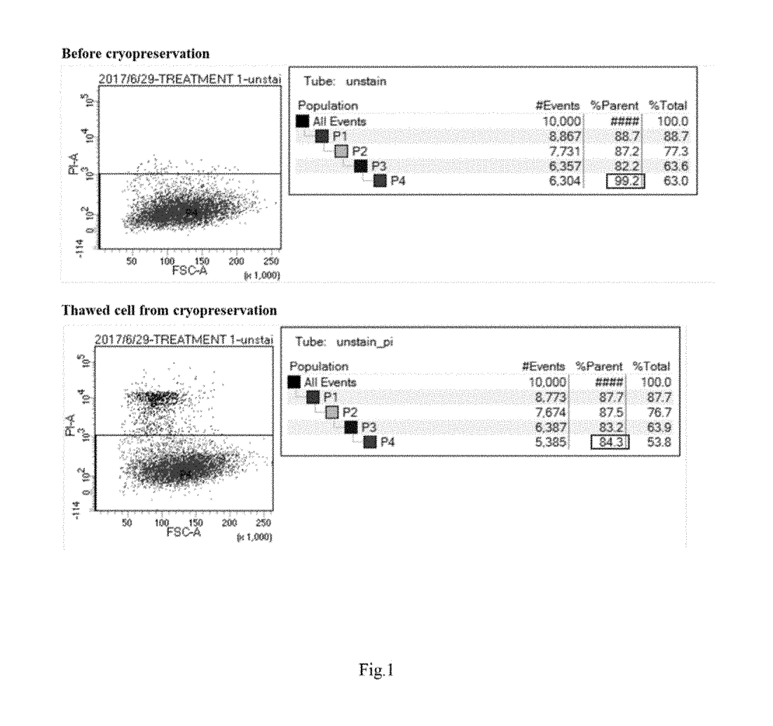

In this example, experiment was performed to further test whether M2C treated by baicalin can express MERTK protein. Using flow cytometry to evaluate macrophage surface protein MERTK expression level in embodiment 1, comparison 1, 2, and the control group is cultured in RPMI 1640 (10% FCS) without any drugs. Result was shown in FIG. 14, after fluorescence staining, fluorescence MERTK marker binding was detected in approximately 80% of cells in every group. However, if use the mean fluorescence intensity (MFI) to calculate the average MERTK expression level in one single cell, as shown in FIG. 1S, M2C macrophages treated with baicalin has significantly two times higher MERTK expression level than other groups (*: compared to control group, #: compared to LPS treated group, ‡: compared to IL-4 treated group, p<0.05). M2C cells treated with baicalin can promote macrophages to express MERTK protein, and the efficacy is greater than LPS and IL-4.

### Example 5: Phagocytosis Promoted by Baicalin in Macrophages

In this example, experiment was performed to evaluate the effect of baicalin induced differentiated M2C macrophages in promoting phagocytosis. Treatment conditions of drugs in each group is listed in chart 2, and control group is cultured in RPMI1640 (10% FCS) without any drugs. After treatment, collect 5×10⁵ treated cells respectively and add 50 μl FITC-Latex beads (Phagocytosis assay kit (IgG FITC), Cayman Chemical, USA.) to perform phagocytosis experiment for 24 hours, and then use PE-anti-MERTK antibody to mark the cells and observe the fluorescence expression with flow cytometry.

FIG. 16 shows the percentage of macrophages phagocytising beads in each group, whereas the blue area indicates the portion of cells that phagocytised the beads. In the group treated by baicalin, approximately 30% of cells phagocytosed the beads and hence fluorescence was detected in flow cytometry. This result was significantly higher than other groups (*: compared to control group, #: compared to LPS treated group, ‡: compared to IL-4 treated group, p<0.05).

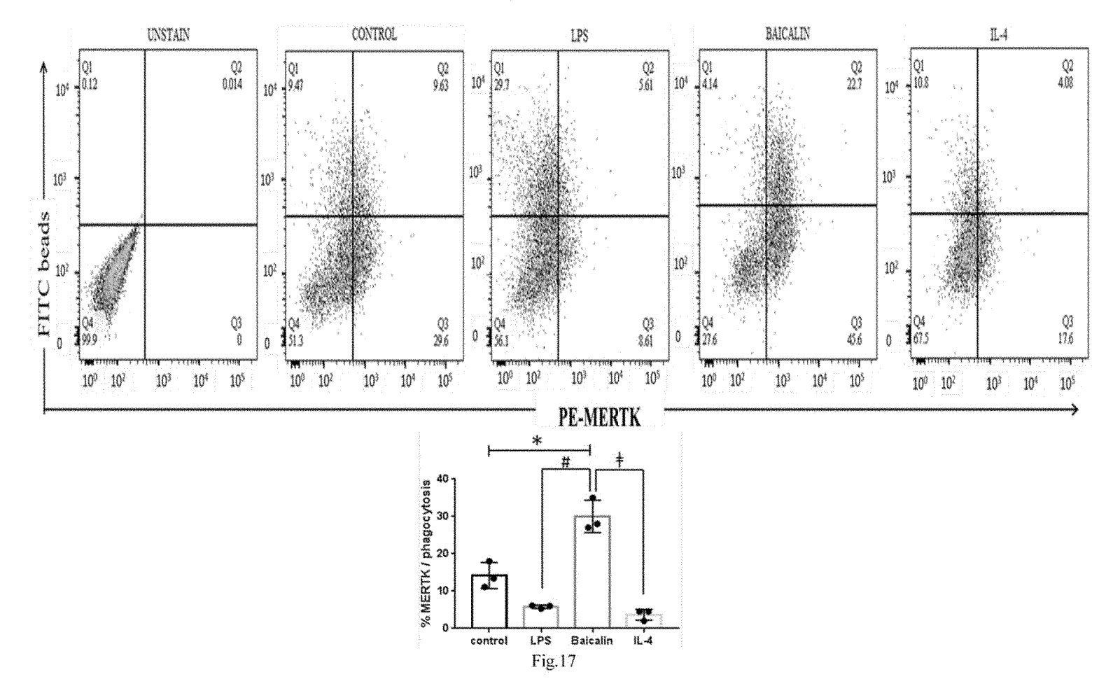

Furthermore, observe the percentage of cells that phagocytosed beads and also express MERTK protein in each group. FIG. 17 showed that 30% of cells treated with baicalin simultaneously express the fluorescence from stained beads and MERTK protein. The result indicates that the group treated with baicalin, most cells that expressed MERTK protein had phagocytosed beads, which means that MERTK protein expression is highly associated with phagocytosis. In other groups, the percentage of MERTK protein expressing cells that phagocytised beads was significantly lower than cells treated with baicalin (*: compared to control group, #: compared to LPS treated group, ‡: compared to IL-4 treated group, p<0.05).

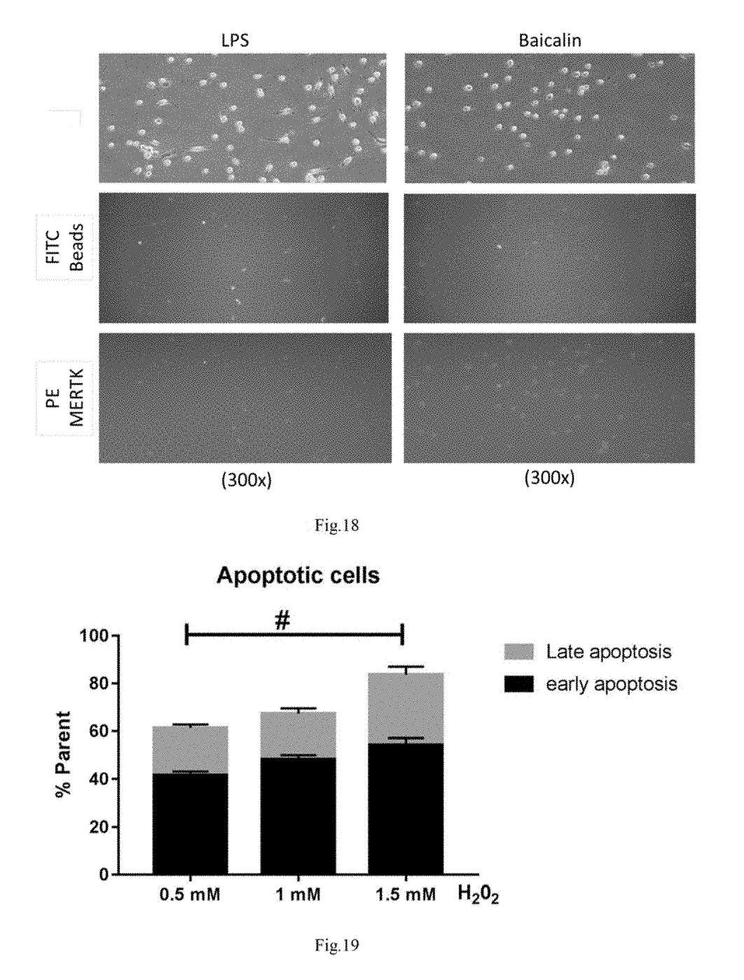

In addition, FIG. 18 shows the fluorescence distribution of cells marked with PE-MERTK after phagocytosing FITC-Latex beads. Baicalin treated macrophages that phagocytosed beads express MERTK on cell membrane, showing red fluorescence light circulated around the cells. On the contrary, MERTK expressed comparatively weaker in group treated with LPS.

Previous studies have shown that LPS can increase phagocytosis capacity by activating the Toll-like receptor-4 on M1 macrophage surface. However, in this example, it is demonstrated that the macrophages which was differentiated by baicalin induction, promoted phagocytosis by enhancing the expression level of MERTK on cell membrane. This new revelation is firstly obtained by the inventor with combination of aforementioned experiments.

### Example 6: Efferocytosis Promoted by Baicalin in Macrophages

In this example, experiment was performed to evaluate the effect of baicalin induced differentiated M2C macrophages on apoptotic cells efferocytosis process, detailed steps are described below.

Inducing apoptotic cells: minor changes were made from steps of obtaining apoptotic cells based on previous studies (Piao et al., 2011 and Fong et al 2007), detailed steps comprising: culture 1.5×10⁶ RAW264.7 macrophages with DMEM medium containing 10% FBS in incubator (37° C., 5% CO₂). After culturing overnight, treat with 0.5, 1, 1.5 mM H₂O₂ respectively in DMEM for 12 hours. Next, remove the DMEM medium containing H₂O₂ and wash the cells with PBS buffer, then use micropipette to transfer cells to 15 ml centrifuge tube and centrifuge cells at 4° C. for 5 mins at rotating speed of 500×g. Remove the supernatant and resuspend the cells with 95 μl P85. Following that, dye the cells with fluorescence marker annexin-V and 7-AAD (BD Bioscience) for 20 mins on ice, avoiding light. After marking the cells, use flow cytometry (FACS BD ARIA II) to detect fluorescence performance. Analysed by software CellQuest (BD Bioscience). If the cells were positive for annexin-V and negative for 7-AAD, the cells will be identified as early apoptotic cells; if the cells were positive for annexin-V and positive for 7-AAD, the cells would be identified as late apoptotic cells. Analytical results in FIG. 19 showed that group treated with 1.5 mM H₂O₂ have a higher percentage of apoptotic cells.

Efficacy evaluation of apoptotic cells efferocytosis in macrophages: method of the evaluation for effectiveness of apoptotic cells efferocytosis in M2 macrophage induced differentiated by different drugs were slightly modified based on previous studies (Li et al., Yancey et al., and Jehle et al; Liu st al and Lilis at al 2008). Detail steps are described below. Use 3 μM CFSE Cell Trace (Thermofisher, USA) to mark RAW264.7 macrophage cell line for 20 minutes under 37° C., then wash with PBS. Culture cells for 24 hours, then use the 1.5 mM H₂O₂ following the aforementioned steps to induce RAW264.7 macrophage cell to differentiate into apoptotic cells. Next, in order to perform efferocytosis, culture both obtained apoptotic cells and M2 cell line from murine bone marrow treated under different drugs (Table.1), with a ratio of apoptotic cells to M2 cell line as 3:2 for 2 hours under 37° C., 5% CO₂. After culturing for 2 hours, wash with PBS and remove unattached cells, then use Trypsin-EDTA to collect attached M2 macrophages. Following that, use PE-anti-MERTK antibody (BioLegend, USA) to mark M2 macrophages while each reaction requires 0.25 ug antibody. Lastly, use flow cytometry (FACS BD ARIA II, BD Bioscience, USA) to examine fluorescence performance, if both PE-MERTK and CFSE appear positive, the cell will be identified as the M2C macrophages that efferocytosed the aforementioned apoptotic cells.

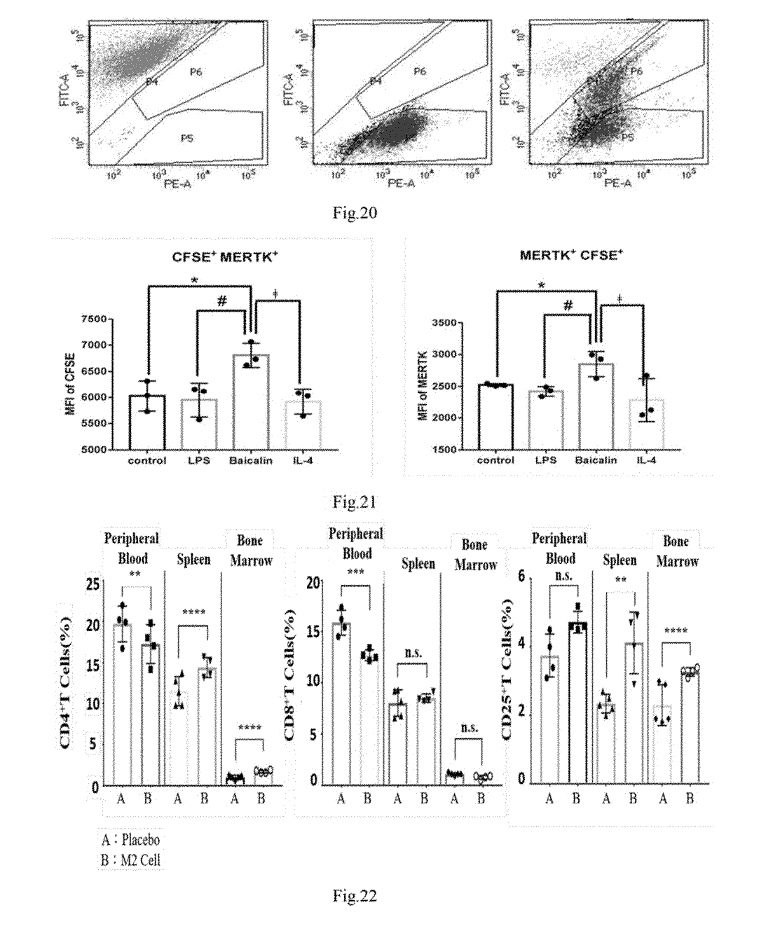

Results of fluorescence expression of stained apoptotic cells, macrophages and efferocytotic macrophages detected by flow cytometry are shown in FIG. 20. Apoptotic cells induced by H₂O₂ (1.5 mM) and express FITC fluorescence after FITC-CFSE staining are distributed in P4 region. Differentiated M2 macrophage expresses MERTK are detected by the expressed PE fluorescence after PE-MERTK staining, which is distributed in P5 region. Macrophages that efferocytosed apoptotic cells are MERTK positive and CFSE positive, which are distributed in P6 region.

After calculating the number of cells in each region, calculate mean fluorescence intensity (MFI) to evaluate average CFSE expression level of cells expressing MERTK, and also evaluate average MERTK expression level of cells expressing CFSE. FIG. 21 shows that the mean fluorescence intensity of MERTK and CSFE of single M2 macrophage that were differentiated by baicalin induction is significantly higher than other drug treated groups and control group (*: compared to control group, #: compared to LPS treated group, ‡: compared to IL-4 treated group, p<0.05). M2C macrophages treated with baicalin can promote macrophages to express MERTK protein, and the efficacy is greater than LPS and IL-4. Therefore, this example further proves that the M2C macrophages differentiated by baicalin induction can promote apoptotic cells efferocytosis by enhancing the expression level of MERTK on cell membrane. The efficacy is significantly higher than macrophages differentiated by IL-4 and LPS drugs induction.

### Example 7: Murine T Cell Immune System Regulation by M2C Macrophage Cell Line

In this example, in vivo experiment in murine was performed to examine the effect of M2C cell line of present invention on T cell immune regulation. Detailed steps are described below.

5×10⁵ Raw 264.7 cells (ATCC®TIB-71) were seeded into a 35 mm culture dish (Corning). After incubating overnight, Raw 264.7 cells were induced with 100 uM Baicalin (TCI, Japan) for 48 hours and incubated at 37° C., 5% CO2. Using Flow cytometry (80 FACSAria II: BD Biocsiences, San Jose, USA) to characterized M2 subtypes with anti-CD11b+(macrophage maker), anti-PD-L1+, and anti-MerTK+ antibodies (Biolegend, San Diego, Calif., USA).

9˜12-weeks-old female C57BL/6 NarI mice were randomly divided into 2 groups: For M2 macrophages treatment group, the mice were transplanted with 1×10⁶/100 μl (cells/volume) RAW 264.7 cells derived M2 macrophage induced by baicalin, using retro-orbital injection on day 0, day 7 and day 14. In parallel for PBS placebo group, 100 μl PBS (placebo) was injected. The mice were sacrificed on day 19 after transplantation and monocytes from peripheral blood, spleen and bone marrow were collected for T-cell composition analysis by flow cytometry.

The whole spleen was gently squeezed by glass grinder and the dissociation of cells was filtered through a 40 μm cell strainer (BO Biosciences, San Jose, USA). 50 μl heparinized blood were lysed with 3 ml RBC lysis buffer (37° C., 15 min) to remove the red blood cells. Total bone marrow cells were collected from single left femur of mice by flushing with 1 ml PBS twice through 25 G syringe.

The monocytes of spleen, peripheral blood and bone marrow were then washed and resuspended with PBS. 1×10⁶ cells were incubated on ice with fluorescein isothiocyanate (FITC-CO25), allophycocyanine (APC-CD4), or phycoerythrin (PE-CD8) antibodies up to total volume of 100l in polystyrene tube. CD4, CD8 and CD25 T-cells composition were analysed by BD FACSAria II flow cytometer (BD Biosciences, San Jose, USA). The data analysed by flow cytometry was analysed by statistical software GraphPad Prism 7 (GraphPad) to determine if it is significantly significant between two treated groups (p<0.05).

Results shown as FIG. 22, after injecting M2C cell line in the experimental group (treated with M2C macrophage), the T cells expressing CD4 or CD8 in peripheral blood were significantly lower than the placebo control group, whereas it was higher in spleen and bone marrow. Owing to the main phenotype of cytotoxic T cell is CD8⁺ and the main phenotype of regulatory/suppressor T cell is CD4⁺CD25⁺, this example further proves that after injecting the M2C cell line of this invention for in vivo cell therapy, it can inhibit the cytotoxic T cell function in peripheral blood, increase regulatory T cells in bone marrow/spleen and promote tissue autoimmune tolerance.

### Example 8: Evaluation of Dermal Fibroblasts Proliferation Promotion by Baicalin-M2C Macrophage Conditioned Medium

After euthanizing the mice, collect skin tissue from infra-axillary of mice and culture the tissue fragments in 37⁰c in DMEM/F12 medium (contains 15% FBS 1× antibiotic/antimycotic) containing digestive enzyme Uberase Blendzyme 3 (0.14 Wunsch units/mL, Roche, Switzerland). Wait until the fluid becomes cloudy and edges of the pieces become fuzzy, then break up the tissue cluster by pipetting continuously and terminate digestive enzyme process in 37° c. DMEM/F12 complete medium. Next, centrifuge mixture under 524×g for 5 minutes and remove the supernatant. Resuspend mixture using 37° c. DMEM/F12 complete medium and repeat this cycle more than once to remove the remained digestive enzyme. After removing digestive enzyme, transfer mixture to tissue culture petri dish with DMEM/F12 complete medium and incubate it in incubator under 37° c., 5% CO₂, 3% O₂ r. Since dermal fibroblasts starts to detach tissue fragments after 2˜5 days and attach to petri dish, it is necessary to observe its attachment condition daily and replace the complete medium when the cell is overgrowing. After confirming all the live dermal fibroblasts have deviated from tissue fragments, dispose the old medium including tissue fragments, and seed the cells by 5×10⁵ cells/plate in new petri dish containing EMEM complete medium (including 15% FBS, 1× Penicillin/Streptomycin, non-essential amino acids, and sodium pyruvate). Because EMEM medium only supplies dermal fibroblasts growth, all other cell types will either cease or stop proliferating.

Seeding for few generations and collect the 5^{th} or 6^{th} generation dermal fibroblasts. Culture cells respectively in baicalin-M2C macrophage conditioned medium and IL-4 M2 macrophage conditioned medium with cell density of 1.5×10⁴ cell/cm². Observe cell proliferation condition after incubating 24, 48, 72 hours.

Cell proliferation is evaluated by observing the CFSE fluorescence reduction and further determining cell doubling time. Detailed steps are described below. Resuspend cells with PBS to obtain cell with cell concentration of 1×10⁶ cell/ml. Add Cell Trace CFSE solution (final concentration 10 μM/ml, Thermo Fisher Scientific, USA) and culture it in 37° c. incubator for 10 minutes for staining. Next, cease CFSE activity with complete medium addition (5 times the volume of CFSE). Stand on ice 5 minutes for reaction, then centrifuge, (500×g, 5 mins), and rinse the cells with fresh complete medium after suspension. Repeat this centrifuge-suspension process 2˜3 times to completely rinse CFSE, then use flow cytometer to analyse the regression of fluorescent cells.

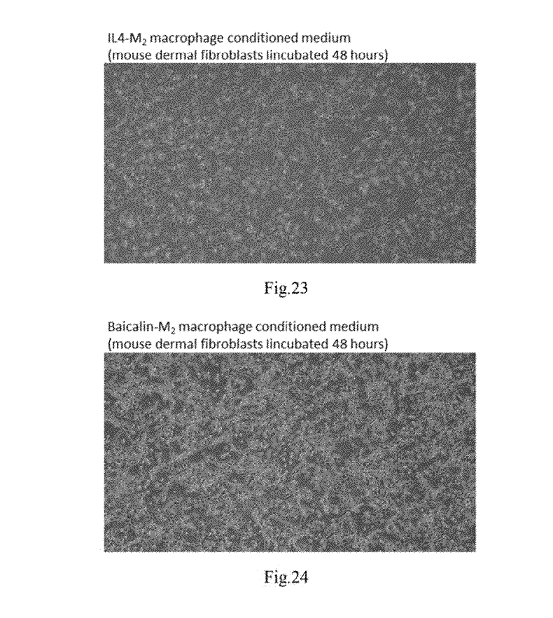

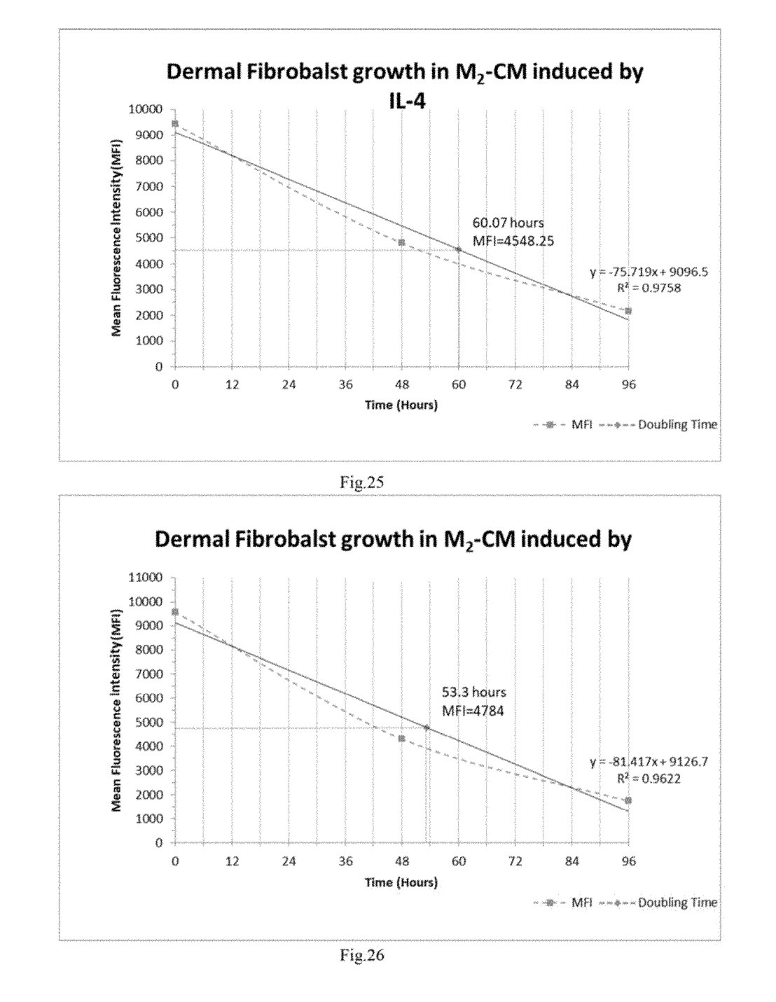

Cell growth condition are shown in FIG. 23 and FIG. 24, after culturing for 48 hours, the murine dermal fibroblasts concentration was higher in baicalin-M2C macrophage conditioned medium comparing to IL4-M2 macrophage conditioned medium. Furthermore, according to results from flow cytometer analysis shown in FIG. 25 and FIG. 26, the doubling time of murine dermal fibroblasts in baicallin-M2C macrophage conditioned medium was 53.3 hours while it was 60.07 hours in IL4-M2 macrophage conditioned medium. This result showed that the baicalin-M2C macrophage conditioned medium prepared on baicalin induced differentiated M2C macrophages can effectively enhance dermal fibroblasts proliferation.

### Example 9: Evaluation of Wound Healing Improvement by Baicalin-M2C Macrophage Conditioned Medium

Use mice (C57BL/6J, 8-20 weeks, Taiwan National Laboratory Animal Center) as experiment model, and anesthetized them with Zoletil 50® (Virbac, France) by intraperitoneal injections. After confirming mice have been anesthetized, remove the hair on back of mice and establish 2 full thickness skin wounds (1.5×1.5 cm²) by excision. Disinfect the wound by 70% ethanol, and then use baicalin-M2C macrophage conditioned medium hydrogels and IL4-M2 macrophage conditioned medium hydrogels respectively as wound dressing to cover the wounds.

Fix the wound with micropore (Soft Cloth Tape®, 3M, USA) and feed the mice in their individual cages. At given point in time (After 3, 6, 9, 12, 15 days), observe the wound using digital camera and estimate the wound by Adobe® Acrobat® 7 Program to calculate the comparative wound healing percentage.

Calculation of comparative wound healing percentages shown below:

Comparative wound reduction percentage (%)=[(A₀−Aₜ)/A₀]×100

A₀: initial wound size.
A₁: wound size after period of time “t”.

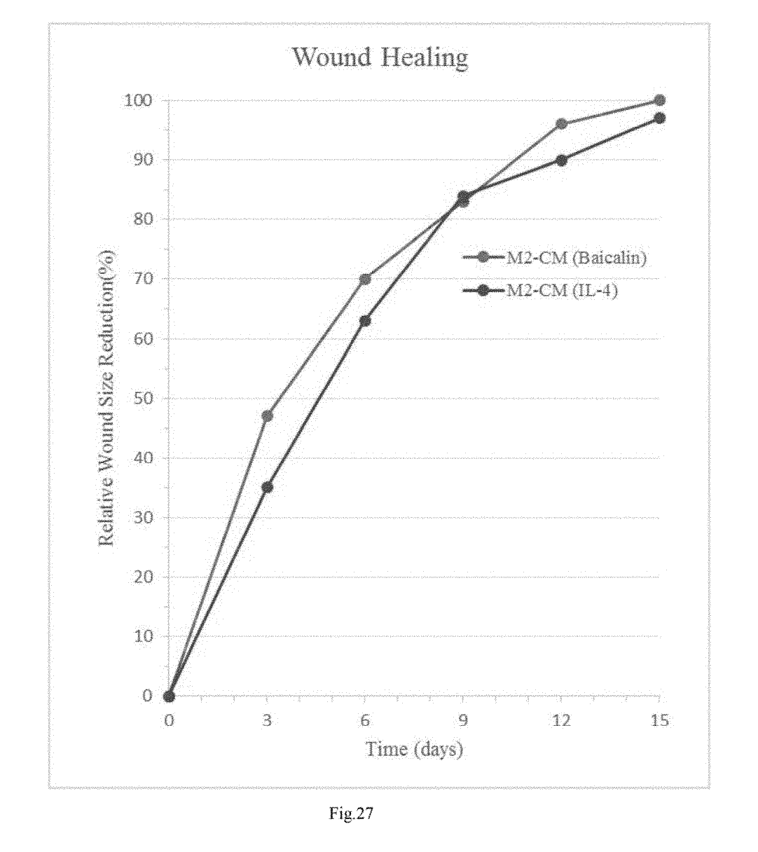

The results after applying wound dressing is shown in FIG. 27, except the measurements on day 9, baicalin-M2C macrophage-conditioned medium hydrogel has greater efficacy of wound healing than IL4-M2 macrophage-conditioned medium hydrogel at any other given point in time.

According to the aforementioned examples and results, it is demonstrate that the baicalin induced differentiated M2C macrophage NPUST-M2φ-1 in this invention has higher MERTK and PTX3 expression level, which can promote phagocytosis such as efferocytosis and further reduce inflammation caused by accumulation of apoptotic cells. Meanwhile, the PD-L1 expression level of M2C macrophage in this invention is also high, which can inactive the cytotoxic T cells in peripheral blood, increase regulatory T cells in bone marrow and spleen, promote tissue autoimmune tolerance and hence help to maintain immune tolerance balance. Moreover, the expression level of VEGF-A and its receptor VEGFR-1, VEGFR-2 of M2C macrophage in this invention is also high, which consequently promote angiogenesis and other functions of VEGF family by autocrine regulation mechanism.

Therefore, the M2C macrophage and polarization induction method by balcalin in this invention is proved to have multiple efficacies and can be applied in cell therapy and other related biopharmaceutical to improve chronic inflammation and autoimmune diseases. In addition, these cell therapies can be applied in human and multiple species in mammals. This invention can be applied in cell therapy, preventive healthcare of specific diseases and many other medical uses. Inventions as such have never been revealed in this technical field, hence the present invention is novel and innovative.

Also, the baicalin-M2C macrophage-conditioned medium in this invention can promote function such as fibroblast proliferation and angiogenesis. The wound dressing containing baicalin-M2C macrophage-conditioned medium prepared from this invention can certainly improve wound healing. It can be used in medical field as well as in skin care products which requires functions such as repair or regenerate. Such technical means mentioned above are not seen in any other invention of this technical field. This invention is novel and innovative.

The above terms and explanations are included but not limited to demonstrate embodiments of the invention. Accordingly, this invention includes all embodiments, modifications and variations that contain technical features of the present invention without departing from the spirit and scope of the invention, and the scope thereof is determined by the appended claims.

What is claimed is:

1. A M2C macrophage cell line called NPUST-M2ϕ-1, deposited in CCTCC (China Center for Type Culture Collection) with accession number C2017269, wherein the M2C macrophage cell is derived from monocytes collected from bone marrows or peripheral blood, and differentiated into M2 macrophage using M-CSF (macrophage colony-stimulating factor), then further induced to differentiate into M2C macrophage using baicalin.

2. A method to prepare a M2C macrophage cell line, comprising:
Step (a): Collecting monocytes from bone marrow and peripheral blood;
Step (b): Using M-CSF to induce M2 macrophage polarization of monocytes;
Step (c): Using baicalin to induce M2C macrophage polarization of M2 macrophage.

3. The method of claim 2 wherein the method is used for cell therapy.

4-20. (canceled)

21. The method of claim 3, wherein the cell therapy is used to promote phagocytosis.

22. The method of claim 3, wherein the cell therapy is used to regulate T cell immunity.

23. The method of claim 3, wherein the cell therapy is used to regulate VEGF gene.

24. The method of claim 2, wherein the method is for preparing phagocytosis-promoting cell preparation.

25. The method of claim 2, wherein the method is for preparing T cell immune-regulating cell preparation.

26. The method of claim 2, wherein the method is for preparing VEGF gene-regulating cell preparation, and the VEGF gene is selected from the group of VEGF-A, VEGFR-1 and VEGFR-2.

27. The method of claim 3, wherein the cell therapy can ameliorate mammal autoimmune diseases.

28. The method of claim 2, wherein the method is for preparing cell preparation for amelioration of mammal autoimmune diseases.

29. A baicalin-M2C macrophage-conditioned medium that improves wound healing, which is obtained after culturing baicalin-induced M2C macrophage in basal medium for 4 hours.

30. The baicalin-M2C macrophage-conditioned medium of claim 29, wherein the basal medium is X-VIVO-10.

31. The baicalin-M2C macrophage-conditioned medium of claim 29, wherein the baicalin-induced M2C macrophage can be obtained from incubating M2 macrophage for 24 hours in medium containing 50 μM baicalin.

32. The baicalin-M2C macrophage-conditioned medium of claim 29, wherein the baicalin-M2C macrophage-conditioned medium is used to prepare a wound dressing that improves wound healing.

33. The baicalin-M2C macrophage-conditioned medium of claim 32, wherein the substrate of the dressing is polyvinyl alcohol/dextran hydrogel.

34. The baicalin-M2C macrophage-conditioned medium of claim 32, wherein the dressing is used to promote fibroblast proliferation and angiogenesis.
